(19) United States
(12) Patent Application Publication
(10) Pub. No.: US 2011/0092909 A1
(43) Pub. Date: Apr. 21, 2011

(54) DEVICE FOR SURGICAL OPERATION OR ULCERATION

(75) Inventors: Morgan Andersson, Lit (SE); Matts Andersson, Lerum (SE)

(73) Assignee: ORTOMA AB, GOTEBORG (SE)

(21) Appl. No.: 11/997,910

(22) PCT Filed: Jul. 27, 2006
(86) PCT No.: PCT/SE2006/000916

§ 371 Date: Sep. 25, 2008

(30) Foreign Application Priority Data
Aug. 5, 2005 (SE) 0501785-0

(51) Int. Cl. A61B 17/34

(52) U.S. Cl. 604/164.04

(57) ABSTRACT

A device is used for expanding a surgical operation or an ulceration, particularly a deep ulceration, and for maintaining it in expanded condition. The device incorporates a spiral and a cloth-formed material associated thereto. The spiral and the cloth-formed material are arranged to form an internal passage, which makes possible access to inner part of the human body, e.g. a vertebrate, vein, intestine, etcetera. The device is easy to install and can he adapted to the type of operation or wound. It is made in technically simple material and I advantageous from cost aspects.

The present invention refers to a device for expanding a surgical operation or an ulceration, particularly a deep ulceration, and for maintaining it in expanded condition.

In connection to surgical incisions or deep wounds which are made or which has occurred in or upon the human body, it is earlier known to use grab scissors or pincers for pressing the edges of the incision or the wound to the sides and thereby open a passage from the external side of the human body to that or those organs to which access inside the human body is wanted.

There is a demand for an instrument, device or implement which simplifies the forming of the passage and prevents flesh and muscle remainders or excesses from penetrating into the passage during the treatment, e.g. the surgical work, inside the human body. There might also be a demand for one time use of said implement which is used for the forming of the passage. Thus there is a requirement that the device for forming and maintaining the passage can be designed in a technically simple manner, but still be functioning well. In some instances there might also be a desire in adapting the device to certain types of operations and/or wounds. Thus there is a requirement to be able to design the device for different types of operations and wounds and in such a manner making the device available in different designs, e.g. conical, as a frustrum of a cone, cylindrical, etcetera. The device shall be designed thus that it can be screwed or pushed into the wall of the operation or the wound. The purpose of the present invention is to solve the entire or parts of this complex of problems.

The thing which can be considered mainly characterizing for a device according to the invention is i.e. that it incorporates a spiral and for this a cloth formed material, whereby the spiral and the cloth formed material together form an internal passage.

In further developments of the inventive idea the cloth formed material can be affixed to the inner and/or outer sides or outer sides of the spiral. The device might substantially have the shape of a cone, a frustrum of a cone or a cylinder. The cloth formed material or materials can furthermore be arranged for preventing penetration of solid body products in the internal passage formed by the device. The spiral and the cloth material on one embodiment might be designed for allowing compressive motions from an initial position. The spiral and the cloth formed material can be designed for permitting that they are screwed in or pushed into the surgical operation or the wound. In one embodiment the spiral can be designed to give a thread cutting function or to have a thread, which at application of the spiral into the incision or the wound begins to co-operate with the walls of the incision or the wound during its screwing function. The cloth material can be affixed on the outer side and/or inside the spiral by means of molding.

By means of the things proposed here above it is possible to achieve an efficient edge pressure on the edges of the surgical incision or the wound. Penetration into the passage of muscle and flesh edges can be efficiently prevented and also substantial penetration of body fluids into the passage. The spiral can be made in a plastic material and the cloth material may also be made in plastic material. In such a manner the device can consist of cost-effective material and form disposable products. The device can be adapted to the type of incision or wound and thus it is possible to make devices of different construction available on the market.

An embodiment proposed at present and which presents the characteristics significant to the invention shall be described hereinafter under reference to the accompanying drawings, where

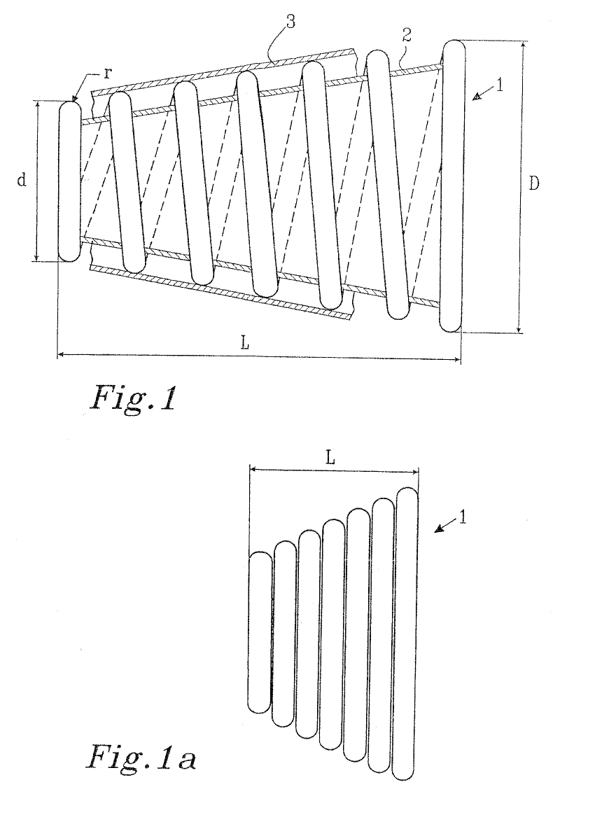

FIG. 1 in a side elevation shows a spiral with the shape of a frustrum of a cone and at or on which has been arranged cloth material on the outside of the spiral and/or the outer and inner sides of the spiral,

FIG. 1a a sketch illustrating the principle the spiral in compressed state in relation to the situation in accordance with FIG. 1,

FIG. 2 shows in an end view the spiral according to FIG. 1 from below,

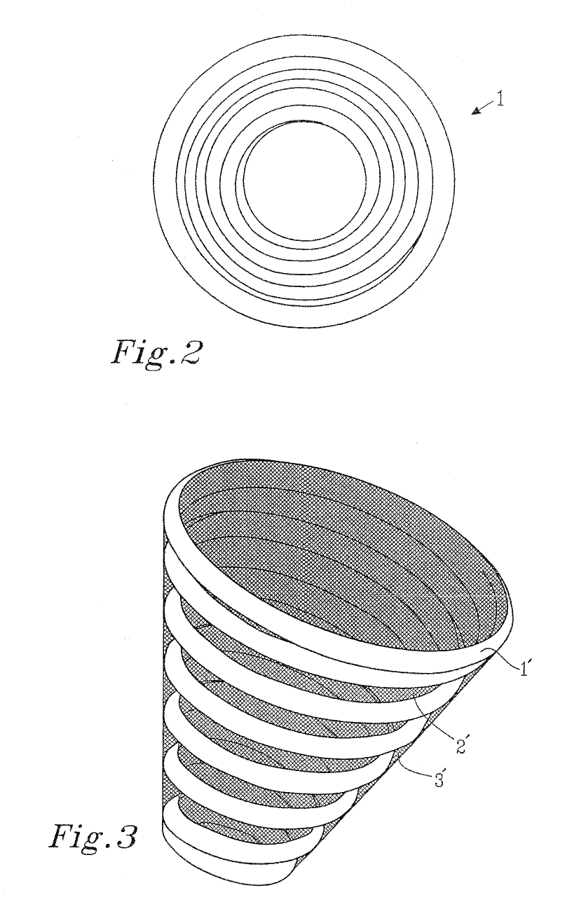

FIG. 3 shows in perspective obliquely from below a structural design of the spiral with cloth material intimated and fitted in symbolic form,

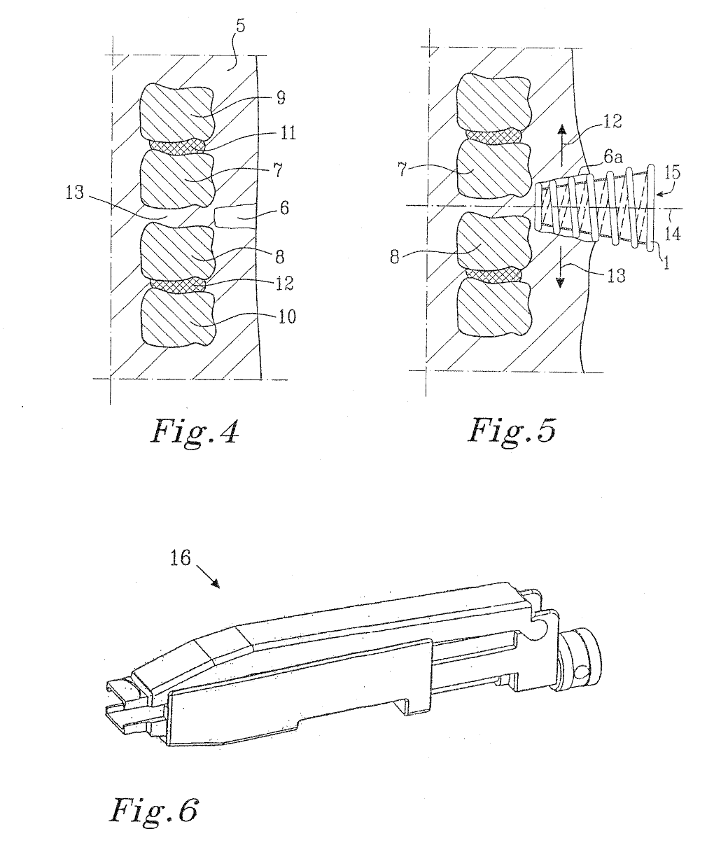

FIG. 4 shows a vertical section an example of a surgical operation in connection to a partly shown vertebral column with vertebras,

FIG. 5 shows in a vertical section the device arranged in the incision or the wound, and which urges away the flesh and muscles and forms an internal passage for a tool, and

FIG. 6 shows in perspective obliquely from above an embodiment of a tool, which can be used for a separating action on vertebras in a vertebral column via a passage established by the device according to FIG. 5.

In FIG. 1 the spiral is shown at 1. In the embodiment shown the spiral has the shape of a frustrum of a cone. The spiral might also be designed in other shapes and might be cylindrical, have a convex or concave curvature on the frustrum of a cone, etcetera. The spiral in the embodiment is made of plastic material. Alternatively the spiral can be made from other material, e.g. metal such as aluminum, steel, etcetera. The spiral is provided with a cloth formed material, which can extend either over the inner side or the outer side of the spiral. Alternatively the cloth material may be provided both on the inner side and on the outer side. In Fig. a cloth provided on the inner side of the spiral has been symbolized with 2, and a cloth provided on the outer side of the spiral with 3. In FIG. 1 the cloth material is illustrated with exaggerated thickness for the sake of clarity. The cloth material 2 or 3 has a thickness t, which can be in the range of 0.02-0.1 mm. The material in the spiral is made so that the device fundamentally is compressible from a maximum length L, which can take up values between 150-170 mm. The length L can not be increased, but reduced, which is shown in FIG. 1a. which intends to show the spiral compressed and having a length L′, which is shorter then the length L in FIG. 1. Due to this compressibility it is possible to moderate for different depths and adaptations of the spiral when this is positioned in a current incision or wound.

FIG. 2 shows the spiral according to FIG. 1 in an end view from the lower side of the spiral.

In FIG. 3 is shown a structural design of the spiral with cloth fitted thereto. In this case the spiral is designated 1′ and a cloth provided inside the spiral is designated 2′. In accordance with the above, it is possible to attach the cloth on the outer side of the spiral, which has been symbolized with 3′, which shows an outside cloth in exaggerated thickness for the sake of clarity. The different cloth materials can be affixed to the spiral e.g. in a molding process which can be of according to a known method.

FIG. 4 shows in principle a portion of the human body 5. In this case the device shall be used at a surgical operation 6 in connection to a partially shown vertebral column, four vertebras 7, 8, 9 and 10 of which are shown. Between the vertebras 7 and 9 and between the vertebras 8 and 10 has been shown intermediate membranes 11 and 12. In the space 13 between the vertebras 7 and 8 the membrane has been damaged or vanished and shall be filled with resilient material of artificial character, which is not shown in FIG. 4. The vertebras 7 and 8 fundamentally engage each other before the surgical operation 6 has been made. In the case shown the patient might have been subjected to stretching, which has resulted in said space 13. According to the embodiment shown it shall be possible to position an implement between and against the vertebras 7 and 8 in order to widen the space 13 further and make possible insertion of the resilient artificial material.

FIG. 5 shows the case where the spiral according to FIG. 1 has been inserted in the surgical incision 6 and urged the side wall 6a of the incision towards the sides in the directions of the arrows 12 and 13, i.e. substantially radially to the sides as seen from the longitudinal axis 14 of the device. In FIG. 5 the inner passage formed by the device 1 has been shown with an arrow 15.

FIG. 6 shows an example of an instrument or a tool 16, which can be inserted via the passage 15 against the vertebras 7 and 8 for a separating action on these. The instrument or the tool 16 is described in detail in a patent application filed at the same date as the present application. This application has got the title “Anordning för applicering av dämpningseffektuerande material mellan kotor”. Therefore the instrument will not be described in detail herein.

It is understood that the spiral 1 with the associated cloth material can be used for other surgical operations on the human body than the one shown or on wounds, which particularly are deeper. The spiral with the associated cloth material can be easily inserted and in one embodiment, the spiral may be equipped with a not shown cutting edge at its outer side, via which edge the spiral can be threaded down into the human body, e.g. in harder muscle tissue.

The invention is not limited to the embodiment shown above as an example but it can be subjected to modifications within the scope of the following claims and the inventive idea.

1. Device for expanding a surgical operation or a ulceration, particularly a deep ulceration, and for maintaining it in expanded condition,
characterized therein,
that it incorporates a spiral and a cloth-formed material associated thereto arranged to form an internal passage.

2. Device as claimed in claim 1, characterized therein,
that the cloth-formed material is affixed to the inner and/or outer side(s) of the spiral.

3. Device as claimed in claim 1, characterized therein,
that it substantially has the shape of a cone, a frustrum of a cone or a cylinder.

4. Device as claimed in claim 1, characterized therein,
that the or those cloth-formed material(s) are arranged to prevent penetration of solid body products in the internal passage formed by the device.

5. Device as claimed in claim 1,
characterized therein,
that the spiral and the cloth material are arranged to permit compression movement but not pulling-apart movement.

6. Device as claimed in claim 1, characterized therein,
that its spiral and cloth-formed material are arranged for permitting that they are screwed or pushed into the surgical operation or in the wound.

7. Device as claimed in claim 1, characterized therein,
that the spiral is arranged to cause a thread cutting function in the wall of the operation or the wound during its screwing down function therein.

8. Device as claimed in claim 1, characterized therein,
that the cloth material is molded to the inner side and or on the outer side of the spiral.

9. Device as claimed in claim 1, characterized therein,
that the cloth material has the shape of a net with meshes within the range 0.02-0.1 mm.

10. Device as claimed in claim 1, characterized therein,
that the spiral in the case of a frustrum of a cone is provided with a value on its brad base exceeding a value of the small base 3-4 times, and that the spiral has a length of 150-170 mm and a radius for each spiral of 4-8 mm.

11. Device as claimed in claim 2, characterized therein,
that it substantially has the shape of a cone, a frustrum of a cone or a cylinder.

12. Device as claimed in claim 2, characterized therein,
that the or those cloth-formed material(s) are arranged to prevent penetration of solid body products in the internal passage formed by the device.

13. Device as claimed in claim 3, characterized therein,
that the or those cloth-formed material(s) are arranged to prevent penetration of solid body products in the internal passage formed by the device.
